(19) United States
(12) Patent Application Publication
(10) Pub. No.: US 2019/0083745 A1
(43) Pub. Date: Mar. 21, 2019

(54) Adapter Manifold for Aseptic Catheter Injections

(71) Applicants: Keinan Smith, Dallas, TX (US); Casey Murray, Edmond, OK (US); Carter Wright, Tulsa, OK (US)

(72) Inventors: Keinan Smith, Dallas, TX (US); Casey Murray, Edmond, OK (US); Carter Wright, Tulsa, OK (US)

(73) Assignee: Protosynthesis Design, LLC, Edmond, OK (US)

(21) Appl. No.: 16/134,130

(22) Filed: Sep. 18, 2018

Related U.S. Application Data
(60) Provisional application No. 62/560,162, filed on Sep. 18, 2017.

(51) Int. Cl. A61M 25/00

(52) U.S. Cl. CPC A61M 25/0097; A61M 2025/0019; A61M 25/0043

(57) ABSTRACT

A catheter manifold device includes one or more inlet ports and a discharge port. Each port has a housing with a height. Each port also includes a fitting with a height that is less than the housing height. The fitting for each port is concealed from contamination within the port housing. The catheter manifold device also includes a port channel extending from the inlet fitting to the discharge port. Where the catheter manifold has more than one inlet port, each inlet port has a channel. The channel of additional inlet ports may extend from the additional inlet port fitting, to the first port channel, and then to the discharge port. The fittings of the catheter manifold device may be connected to various external devices to protect the fittings or to allow the administration of fluids to a patient.

### CROSS REFERENCE TO RELATED APPLICATIONS

This application claims the benefit of and priority to U.S. Provisional Patent Application Ser. No. 62/560,162, filed on Sep. 18, 2017 entitled “Adapter Manifold for Aseptic Catheter Injection,” and incorporates said provisional application by reference in its entirety into this document as if fully set out at this point.

### FIELD OF THE INVENTION

The present invention relates generally to the field of catheter manifold systems, and more particularly, but not by way of limitation, to a catheter manifold system and methods of improving aseptic catheter injections.

### BACKGROUND

Therapeutic solutions, for example, medications, saline, and the like are typically administered to patients using external syringes (a type of infusion device). The saline and medication may be injected directly into the bloodstream using a venous catheter. The catheter typically consists of a cannula (a rigid plastic tube) inserted directly into a vein via the use of a guide needle, and a tubing lead with a fitting, or termination, on its opposing end. The fitting is used as a port to connect external drug delivery devices including syringes, infusion pumps, and other infusion devices.

The most common method used to inject therapeutic solutions to the body via the use of a venous catheter is called the saline-administration-saline (“SAS”) method. The SAS method consists of three injection steps, the injection of saline to prime the injection port and cannula, then the injection of medication, and, finally, the injection of additional saline to ensure the delivery of the full volume of medication into the vein. The catheter may include terminations, for example, luer lock fittings, allowing them to connect to the fitting (port) on the catheter. Both the fitting on the catheter and the termination on each of the syringes or other infusion device must be scrubbed with alcohol pads to ensure aseptic conditions throughout the SAS process. The catheter fitting has to be scrubbed before the attachment of each syringe or infusion device as does the termination of the syringe or other infusion device being installed. Thus, the SAS method requires at least six scrubbing steps to ensure sterility.

The numerous scrubbing and action steps necessary to transition from using the various syringes or other infusion devices present a major challenge with regards to reducing the risk of a catheter-related bloodstream infection (“CRBSI”). Moreover, causes of contamination presented by the SAS method include various health risks, such as (1) inadequate scrubbing of the infusion device terminations and catheter port by a healthcare worker or other individual administering the medicine, (2) inadequate disinfection of the healthcare worker's gloves and subsequent inadvertent contact with the infusion device terminations or catheter port after the prior scrubbing step has been sufficiently completed, and (3) incidental contact between the infusion device terminations or catheter port with other external objects while syringes or pumping devices are being changed after the prior scrubbing step has been sufficiently completed.

Accordingly, there continues to be a need for a reducing the risk of infections caused by the SAS infusion process. There is also a need for a system to eliminate contamination of the injection port of a catheter by incidental contact during intravenous injections using the SAS method. Further, there is the need for a system capable of connecting multiple syringes or infusion pumps simultaneously to circumvent the need to connect, disconnect, and reconnect fluid inputs. There is the need for a system that reduces the number of external separate components required to do an intravenous injection or flush using the SAS method with a catheter. There is also a need for a system and method of reducing the number of sterilization steps, making it easier for the operator to administer medicine to a catheter patient during catheter injections by an SAS process. There is also a need to reduce the number of times that alcohol pads are required to scrub fittings during catheter injections via the SAS method, to reduce contamination risks. It is to these and other deficiencies in the prior art that the present invention is directed.

### SUMMARY OF THE INVENTION

In one aspect, a catheter manifold device includes an inlet port with an inlet housing, where the inlet housing has a height. The inlet port also has an inlet fitting with an inlet fitting height that is less than the inlet housing height. The inlet fitting is concealed from contamination within the inlet housing. The catheter device also includes a discharge port and a port channel extending from the inlet fitting to the discharge port.

The catheter manifold device may also include a second inlet port with a second inlet housing, where the second inlet housing has a height. The second port includes a second inlet fitting with a second inlet fitting height that is less than the second inlet housing height. The second inlet fitting is contained inside of the second inlet housing and concealed from contamination. The catheter device also includes a second port channel extending from the second inlet fitting to the port channel.

In another aspect, the discharge port includes a discharge housing with a discharge housing height. The discharge port also includes a discharge fitting with a discharge fitting height that is less than the discharge housing height. The discharge fitting is concealed from contamination within the discharge housing.

In another aspect, the catheter manifold device may include a third inlet port. The third inlet port includes a third inlet housing with a third inlet housing height. The third inlet port also includes a third inlet fitting with a third inlet fitting height that is less than the third inlet housing height. The third inlet fitting is concealed from contamination within the third inlet housing. A third port channel extends from the third inlet fitting to the discharge port.

The catheter manifold device may also include a plurality of caps, where each cap has a cap fitting, capable of being connected to one of the inlet, second inlet, discharge, or third inlet fittings. Each cap also includes a cap knob, which extends above the height of a corresponding inlet, second inlet, discharge, or third inlet housings to allow the cap to be removed without contact to the fittings. Once the caps are removed from the various fittings, syringes, catheters and other devices may be connected to the fittings.

In another aspect, a method of administering fluid to a patient, associated with a catheter manifold device, includes connecting a catheter to a discharge port of the catheter manifold device. The method also includes administering a first fluid from a first fluid delivery device. The first fluid flows through a first port channel extending from a first inlet port fitting on a first port of the catheter manifold device into the discharge port, and into the catheter. The method also includes administering a second fluid from a second fluid delivery device, where the second fluid flows through a second port channel extending from a second inlet port fitting on a second port of the catheter manifold device, into the first port channel, into the discharge port, and into the catheter.

The method may also include removing the first fluid delivery device from the first inlet port fitting and connecting a third fluid delivery device to the first inlet port fitting and then administering a third fluid from the third fluid delivery device. The third fluid flows through the first port channel, into the discharge port, and into the catheter.

In yet another aspect, a method of administering fluids to a patient, using a catheter manifold device, includes removing a discharge port cap from a discharge port fitting of a discharge port of the catheter manifold device, and connecting a catheter to the discharge port fitting. The method also includes removing a first inlet port cap from a first inlet port fitting of a first inlet port on the catheter manifold device, where a first port channel extends from the first inlet port fitting to the discharge port.

The method further includes connecting a first fluid delivery device to the first inlet port fitting, and removing a second inlet port cap from a second inlet port fitting of a second inlet port on the catheter manifold device, where a second port channel extends from the second inlet port fitting to the discharge port. The method also includes connecting a second fluid delivery device to the second inlet port fitting, and removing a third inlet port cap from a third inlet port fitting of a third inlet port on the catheter manifold device, where a third port channel extends from the third inlet port fitting to the second port channel, and connecting a third fluid delivery device to the third inlet port fitting

The method also includes administering a first fluid from the first fluid delivery device, where the first fluid flows through the first port channel, into the discharge port, and into the catheter, administering a second fluid from the second fluid delivery device, wherein the second fluid flows through the second port channel, into the third port channel, into the discharge port, and into the catheter, and administering a third fluid from the third fluid delivery device, wherein the third fluid flows through the third port channel, into the discharge port, and into the catheter.

### BRIEF DESCRIPTION OF THE DRAWINGS

FIG. 1 presents a partially exploded perspective view of a catheter manifold system in accordance with a preferred embodiment.

FIG. 2 presents a partial cross sectional view of a catheter manifold device in accordance with a preferred embodiment.

FIG. 3 depicts an upper perspective view of the catheter manifold device of FIG. 2.

FIG. 4 depicts a lower perspective view of the catheter manifold device of FIG. 2.

FIG. 5 presents an exemplary method of installing or removing port caps from the catheter manifold device.

FIG. 6A-6E depict an exemplary method of using the catheter manifold system of FIG. 1.

FIG. 7A-7E depict an alternative exemplary method of using the catheter manifold system of FIG. 1.

FIGS. 8-9 depict a catheter manifold device in accordance with a second preferred embodiment.

### DETAILED DESCRIPTION OF PREFERRED EMBODIMENTS

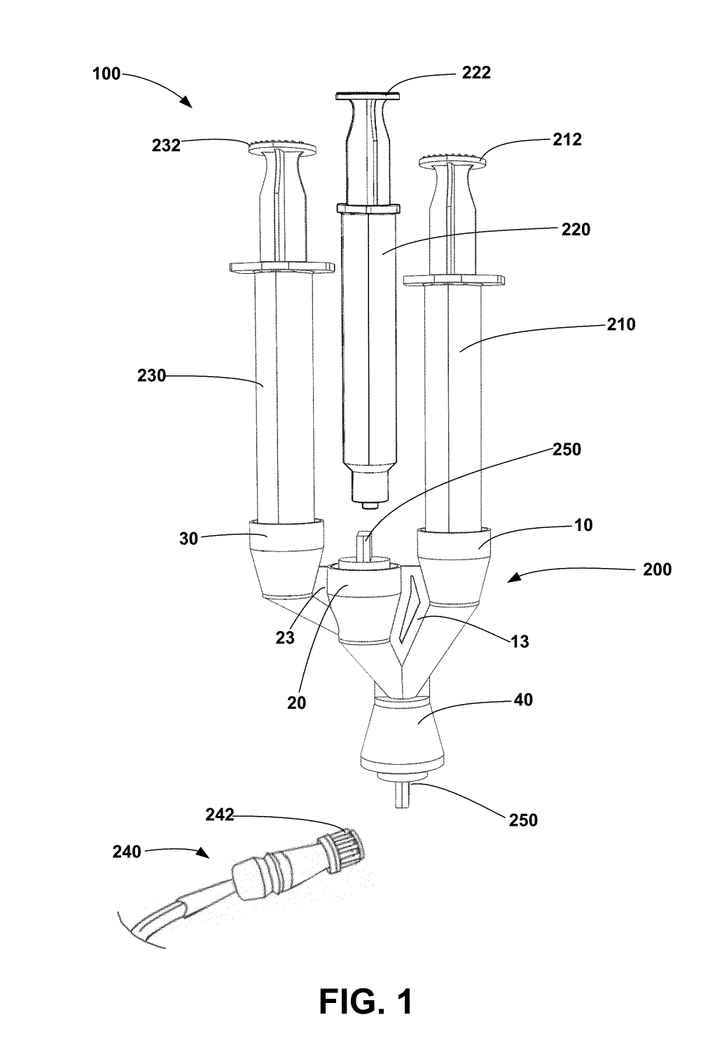

In accordance with an exemplary embodiment, FIG. 1 shows a front perspective view of a catheter manifold system 100. The catheter manifold system 100 includes a catheter manifold device 200. The catheter manifold system preferably includes a wash device 210, a delivery device 220, and a flush device 230. The catheter manifold system may also include a catheter port 240 and a plurality of port caps 250. The catheter manifold device 200 preferably includes a wash port 10, a delivery port 20, a flush port 30, and a discharge port 40.

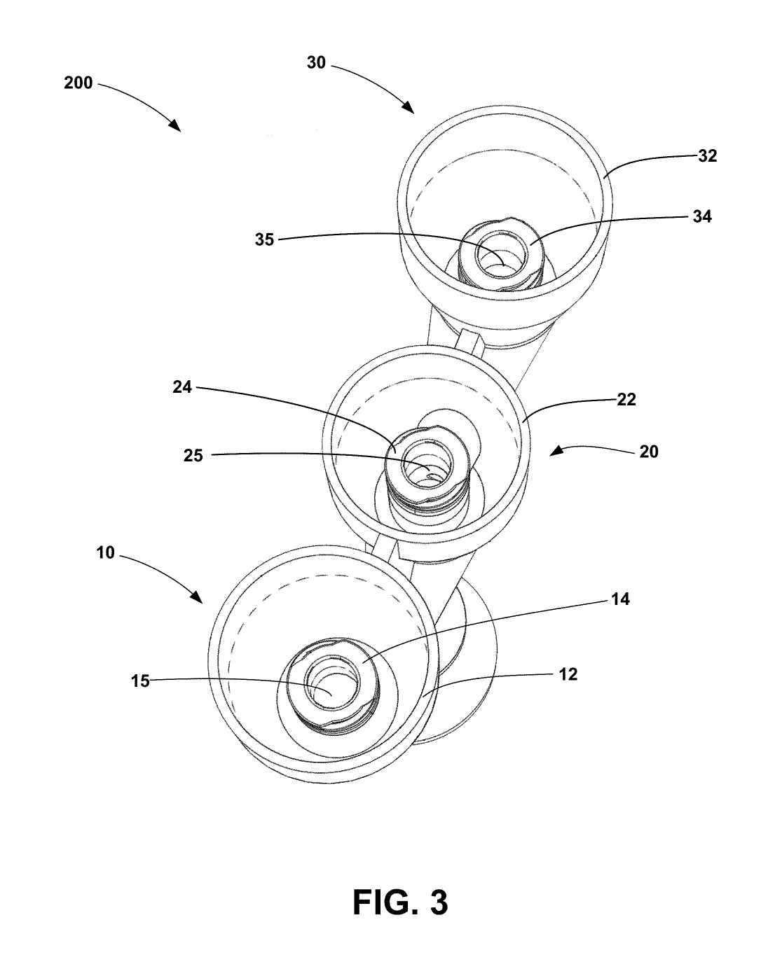

As further depicted in FIGS. 1-3, the wash port 10 of the catheter manifold device 200 includes a wash contamination shield 12, a wash fitting 14, a wash inlet opening 15, a wash channel 16 connected to the wash inlet opening 15 and forming a wash funnel 17, a wash funnel housing 18 surrounding the wash funnel 17, and a wash outlet opening 19.

The wash fitting 14 may be connected to external pumping mechanisms, such as the wash device 210. Alternatively, the wash inlet fitting may be fitted with protective devices, such as one of the plurality of port caps 250. The plurality of port caps 250, the wash device 210, or other external components may be attached to the wash fitting 14 via mating terminations on these components.

The wash funnel 17 provides a path for fluid to flow from the wash fitting 14 through the wash inlet opening 15 and then downstream to the wash outlet opening 19 when the wash device 210 is connected to the wash fitting 14 and fluid, such as saline, is pushed through the wash inlet opening 15.

The wash contamination shield 12 completely envelopes the wash fitting 14 to protect and prevent inadvertent physical contact and contamination of the wash fitting 14. The wash contamination shield 12 is constructed to be a rigid physical barrier capable of reducing the risk of physical contact with components enclosed within the barrier, such as the wash fitting 14. This also reduces the need to scrub the wash fitting 14 prior to use of the catheter manifold device 200.

Also depicted in FIGS. 1-3, the delivery port 20 includes a delivery contamination shield 22, a delivery fitting 24, a delivery inlet opening 25, a delivery channel 26 connected to the delivery inlet opening 25 and forming a delivery funnel 27, a delivery funnel housing 28 surrounding the delivery funnel 27, and a delivery outlet opening 29.

The delivery fitting 24 may be connected to an external pumping mechanism, such as, the delivery device 220, or one of the plurality of port caps 250. The plurality of port caps 250, the delivery device 220, or other external components may be connected to the delivery fitting 24 via mating terminations on these components.

The delivery funnel 27 provides a path for a fluid, such as medicine, liquid diets, or other desired fluids, to flow from the delivery fitting 24 through the delivery inlet opening 25 and then downstream to the delivery outlet opening 29 when an external delivery device 220 is connected to the delivery fitting 24 and fluid is pushed through the delivery inlet opening 25.

The delivery contamination shield 22 completely envelopes the delivery fitting 24 to protect and prevent inadvertent physical contact with the delivery fitting 24. The delivery contamination shield 22 is a rigid physical barrier capable of reducing the risk of physical contact with components enclosed within the barrier.

The flush port 30 includes a flush contamination shield 32, a flush fitting 34, a flush inlet opening 35, a flush channel 36 connected to the flush inlet opening 35 and forming a flush funnel 37, a flush funnel housing 38 surrounding the flush funnel 37, and a flush outlet opening 39. The flush outlet opening 39 is connected to and creates a fluid path downstream into the delivery funnel 27.

The flush fitting 34 may be connected to an external pumping mechanism, such as, the flush device 230, or one of the plurality of port caps 250. The plurality of port caps 250, flush device 230, or other external components may be attached to the flush fitting 34 via mating terminations on these components.

The flush funnel 37 provides a path for fluid to flow from the flush fitting 34 through the flush inlet opening 35 and then downstream to the flush outlet opening 39 when an external flush device 230 is connected to the flush fitting 34 and fluid, such as saline, is pushed through the flush inlet opening 35.

The flush contamination shield 32 completely envelopes the flush fitting 34 to protect and prevent inadvertent physical contact with the flush fitting 34. The flush contamination shield 32 is a rigid physical barrier capable of reducing the risk of physical contact with components enclosed within the barrier.

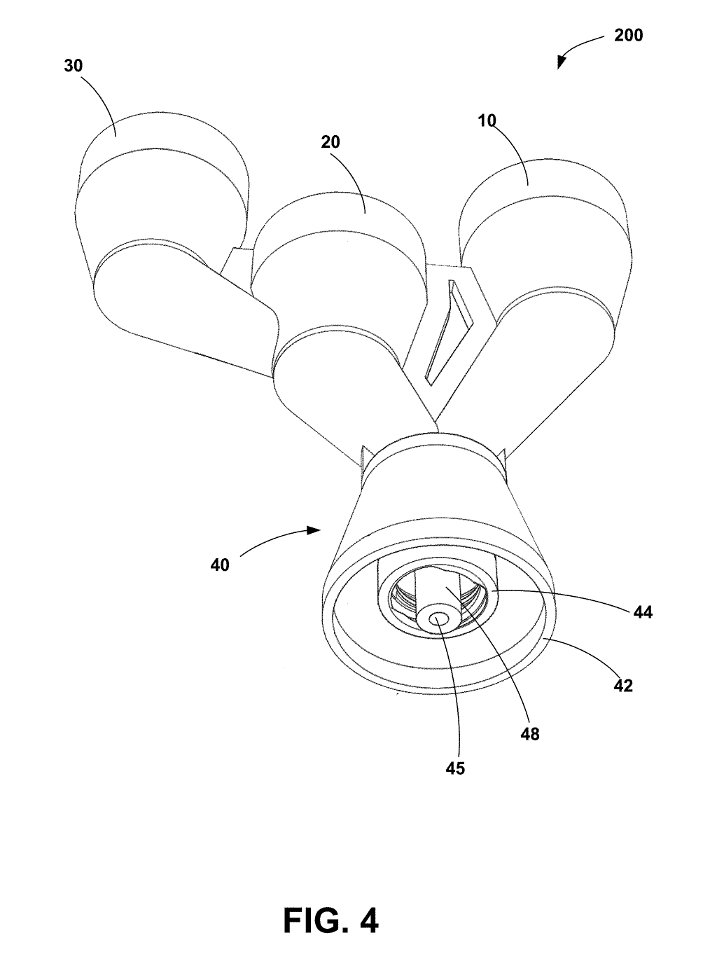

As depicted in FIGS. 1, 2 and 4, the discharge port 40 includes a discharge contamination shield 42, a discharge fitting 44, and a discharge channel 46 connected downstream of the wash outlet opening 19 and the delivery outlet opening 29, and a discharge channel housing 48 surrounding the discharge channel 46. The discharge port 40 also includes a discharge opening 45, positioned downstream from the discharge channel 46.

The discharge channel 46 provides a path for fluid to flow from the wash outlet opening 19 and the delivery outlet opening 29, into the discharge channel 46 and then downstream through the discharge opening 45 and into an external devices, such as the catheter port 240.

The discharge contamination shield 42 completely envelopes the discharge fitting 42 to protect and prevent inadvertent physical contact with the discharge fitting 42. The discharge contamination shield 42 is a rigid physical barrier capable of reducing the risk of physical contact with components enclosed within the barrier.

The discharge fitting 44 may be connected to external devices, for example, the catheter port 240, or one of the plurality of port caps 250. The plurality of port caps 250, catheter port 240, or other external components may be attached to the discharge fitting 44 via mating terminations on these components.

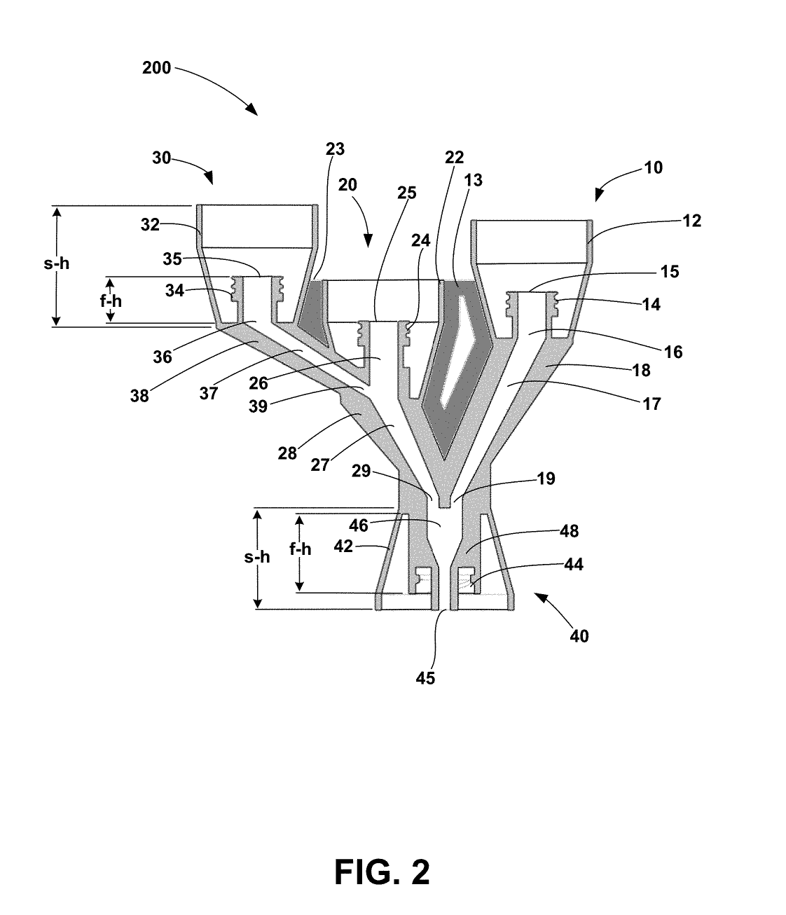

As depicted in FIG. 2, each of the contamination shields (12, 22, 32, 42) each have a shield height (s-h) and each of the fittings (14, 24, 34, 44) has a fitting height (f-h). Although only the shield heights s-h and fitting heights f-h are shown for the flush contamination shield 32, flush fitting 35, discharge contamination shield 42 and discharge fitting 44, it will be understood that the wash contamination shield 12, the wash fitting 14, the delivery contamination shield 22, and the delivery fitting 24 each have corresponding shield heights and fitting heights. Preferably, the fitting heights are less than the shield heights for all of the ports to protect the fitting from contamination within the contamination shields.

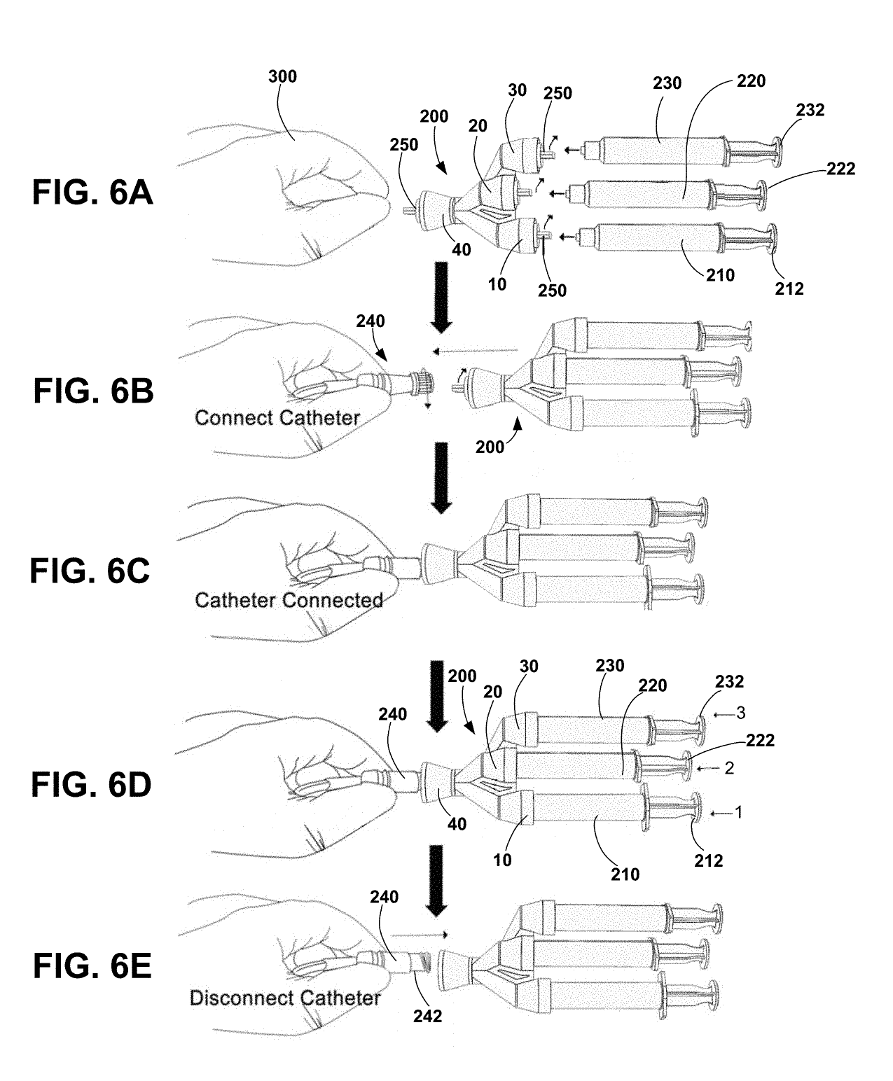

In a preferred embodiment, syringes are shown for the wash device 210, the delivery device 220, and the flush device 230. Syringes are commonly used in the medical industry and are known to be capable of holding fluids, such as saline solutions, medicine or other solutions. Syringes are known to include a mechanical plunger (depicted as 212, 222 and 232 in FIGS. 1 and 6) that, when depressed pushes the fluid within the syringe such that it exits the syringe and flows to an external source.

It will be understood that while syringes are depicted for the wash device 210, the delivery device 220, and the flush device 230, other known devices may be used, such as saline or medicine bags or containers which may be connected via a catheter instead of a syringe. Alternatively to syringes, calibrated electronic pumps may be used which have self-contained automated pumping mechanisms.

In a preferred embodiment of the catheter manifold system 100, a catheter 240 with a catheter port 242 is depicted as being used to connect to the discharge port 40. Catheters are commonly used in the industry to connect various medical devices to a patient via a catheter line. Catheters typically have a cannula, or rigid plastic tube, which is inserted directly into the vein of a patient via the use of a guide needle, and a tubing lead with a fitting (termination), such as the catheter port 242, on the opposing end. The fitting is used as a port to connect external drug delivery devices, such as syringes, infusion pumps and other infusion devices.

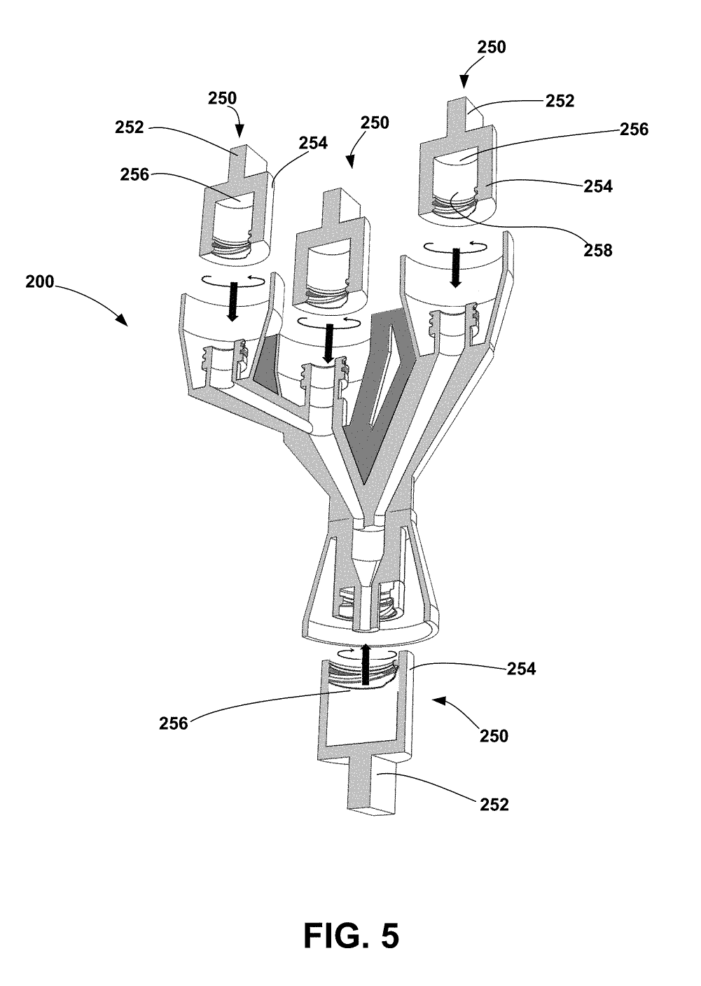

Turning to FIG. 5, depicted therein is a partial cross sectional exploded view of a method of connecting or disconnecting the plurality of port caps 250 onto the catheter manifold device 200. The plurality of port caps each include a port cap knob 252, a port cap shell 254 and an internal space 256 formed within the port cap shell 254. Each of the port caps 250 may include a sterile pad 258 that may be located on within the internal space 256.

The plurality of port caps 250 are used to maintain an aseptic environment within the internal structure of the catheter manifold device 200. The plurality of port caps 250 maintain aseptic conditions by shrouding and protecting the inlet and discharge fittings (14, 24, 34, 44) within the respective ports (10, 20, 30, 40) from inadvertent physical contact when external objects or devices (such as the wash device 210, delivery device 220, flush device 230, or catheter port 240) are not connected to the ports, as shown in FIG. 5.

As depicted in FIG. 5, the plurality of port caps 250 have a height that is more than that of the various port contamination shields (12, 22, 32, 42) such that the port cap knobs 252 extend out of the ports to allow the port caps 250 to be removed without inserting anything within the contamination shields. This feature allows the port caps 250 to be connected and disconnected from the various inlet and discharge fittings (14, 24, 34, 44) by turning the port cap knobs 252 and removing or installing the caps. Because of the height of the contamination shields and the height of the port caps, the risk of touching the fittings (14, 24, 34, 44) is reduced or eliminated. The sterile pad 258 is configured to contact the fittings (14, 24, 34, 44) when the port caps 250 are on the fittings to maintain aseptic conditions within the invention.

It will be understood that the plurality of port caps 250 may be constructed such that the port cap knob 252, port cap shell 254 and internal space 256 formed within the port cap shell 254 are a unitary body. Each of the plurality of port cap knobs may have various shapes and sizes.

As depicted in FIGS. 1-2, the wash port 10 is connected to the delivery port 20 via a first plastic webbing 13 and the delivery port 20 is connected to the flush port 30 via a second plastic webbing 23. Additionally, the wash port 10 and delivery port 20 are connected to the discharge port 40.

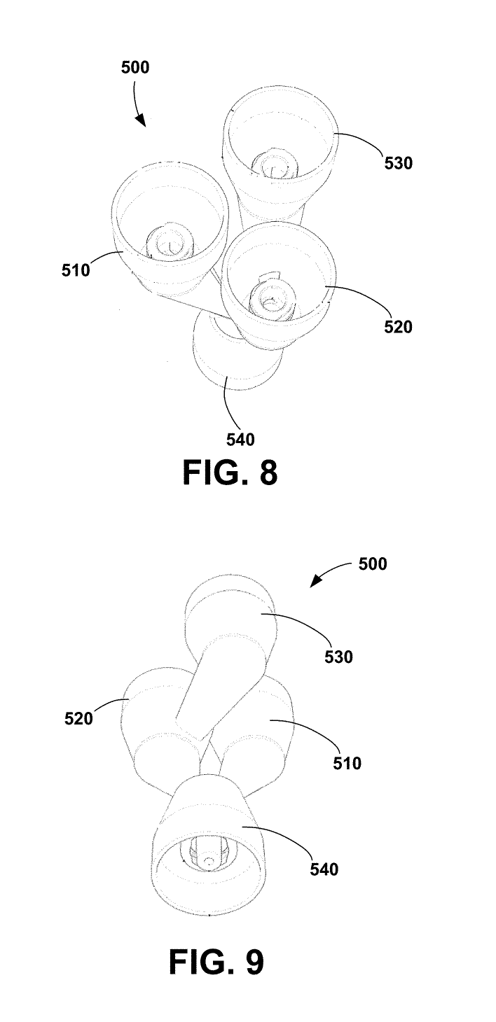

Although FIGS. 1-4 depict a catheter manifold device 200 with a single wash port 10, a single delivery port 20, a single flush port 30 and a single discharge port 40, it will be understood that the catheter manifold device 200 may contain a plurality of each of these ports depending on the desired application. Further, it will be understood that a single port may be used instead of the wash port 10, the flush port 30 and/or the delivery port 20. It will be understood that other ports of the catheter manifold device 200 may be positioned closer or further together, such that the configuration forms a linear type configuration or a staggered type configuration (as depicted in the catheter manifold device 500 depicted in FIGS. 8-9).

Although FIGS. 1-6 depict luer lock connectors for the wash fitting 14, delivery fitting 24, flush fitting 34, discharge fitting 44, the plurality of port caps 250, and the catheter port 240, it will be understood that each of these fittings may be constructed of alternative types and styles of connectors, male or female, such as slip luers, small bore connectors, twist-to-connect couplings, taper seal connectors, tubing connectors, and the like, provided the fitting provides a fluid path for the movement of fluids through the fittings. Depending on the type of connection used, it will be understood that the plurality of port caps 250, the wash device 210, the delivery device 220, the flush device 230, and the catheter port 240 will have the corresponding necessary connections to allow these devices to be secured onto the fittings used.

The catheter manifold device 200 is preferably a unitary molded body, but may alternatively be constructed such that each of the wash port 10, delivery port 20, flush port 30, and discharge port 40 are formed separately and then joined together with known methods. The catheter manifold device 200 is preferably constructed of molded plastic materials, but may also be constructed of other materials, such as metals, rubbers, or the like, where such material preferably provides a rigid structure to prevent contact with, and contamination of, the fittings (14, 24, 34, 44).

Although each of the ports (10, 20, 30, 40) is depicted having a cup like structure, each of the ports may have alternative shapes and sizes and have varying physical dimensions. The size and shape of the various contamination shields, including the wash contamination shield 12, the delivery contamination shield 22, the flush contamination shield 32 and the discharge contamination shield 42 may be of varying sizes to accommodate varying sized syringes and catheter connections. Depending on the size and shape of the fittings and contamination shields, the plurality of port caps 250 may also be altered in size and shape to fit within the contamination shields (12, 22, 32, 42).

The wash funnel housing 18, delivery funnel housing 28, flush funnel housing 38 and discharge channel housing 48 and the corresponding funnels (17, 27, 37) and discharge channel 46 may be of varying sizes and shapes and may vary to adjust for desired fluid flow properties.

When external devices, such as the wash device 210, delivery device 220, or flush device 230, are connected to any one of the wash port 10, the delivery port 20, or flush port 30, fluid may be pushed through the respective openings (15, 25, 35) of the ports and into the respective channels (14, 24, 34). Fluid then travels into the channels (16, 26, 36), through the respective funnels (17, 27, 37), through the respective outlet openings where they are ejected into the discharge channel 46 of the discharge port 40 and may then exit the discharge channel 46 through the discharge opening 45 and into an external device, such as the catheter port 240.

Turning to FIGS. 6A-6E, depicted therein is an exemplary method of using the catheter manifold system 100. As depicted in FIG. 6A, an operator 300 removes the plurality of port caps 250 from the wash port 10, the delivery port 20, and the flush port 30. Preferably after removing the port caps 250, the operator removes any protective cap (not depicted) located on the fittings of the devices (210, 220, 230).

The operator connects the wash device 210 to the wash port 10, the delivery device 220 to the delivery port 20, and the flush device 210 to the flush port 30. These connections may be done in any order.

Turning to FIG. 6B, the operator removes the port cap 250 from the discharge port 40 and then removes any protective cap located on the catheter port 240. The operator connects the catheter port to the discharge port 40 of the catheter manifold device 200 as depicted in FIG. 6C.

Next, as depicted in FIG. 6D, the operator administers the fluids, which may be saline and medicine, to a patient (not depicted), where the patient is connected to an intravenous line, of the catheter port 240. First, the operator administers a fluid, such as saline, using the wash device 210 connected to the wash port 10. The operator administers the fluid by pushing the mechanical plunger 212 of the wash device 210, which pushes the fluid through the wash inlet opening 15 of the wash port 10 and then downstream into the wash channel 14. The saline then travels through the wash stem barrel 17 and through the wash outlet opening 19, into the discharge channel 46, and then exits the discharge opening 45, and preferably travels into the catheter port 240.

After administering the wash fluid, the operator administers a second fluid, such as medicine or other preferred fluids, by pushing the mechanical plunger 222 of the delivery device 220, which pushes the second fluid through the delivery opening 25 of the delivery port 20 and then downstream into the delivery channel 24. The second fluid then travels downstream through the delivery funnel 27, through the delivery outlet opening 29, into the discharge channel 46, through the discharge opening 45, and into the catheter port 240.

After administering the second fluid, the operator administers a third fluid, such as saline by pushing the mechanical plunger 232 of the flush device 230, which pushes the third fluid through the flush inlet opening 35 of the flush port 30 and then into the flush channel 34. The third fluid then travels downstream through the flush funnel 37, through the flush outlet opening 39, into the delivery funnel 27, out of the delivery outlet opening 29, into the discharge channel 46, through the discharge opening 45, and into the catheter port 240. Because the third fluid administered through the flush port 30 flows through the delivery funnel 27 located downstream of the flush funnel 37, the third fluid flushes any of the second fluid remaining in the delivery funnel 27 to ensure that the full dose of the second fluid is delivered to the patient.

As depicted in FIG. 6E, after completing the administration of the third fluid, the operator may disconnect the catheter port 240 from the discharge port 40 and if desired, disconnect the wash device 210, delivery device 220 and flush device 230 from the catheter manifold device 200. It will be understood that in some instances there is no need to disconnect the external devices (210, 220, 230), which may be discarded along with the catheter manifold device 200.

It will be understood that if requested or required, prior to inserting the external devices into the catheter manifold device, the operator may scrub the fittings of the various external devices (210, 220, 230, 240) with a new sterile pad for the required amount of time and lets the fittings dry. It will be understood that various time frames, procedures, and requirements will be made for the operator based on hospital regulations, policies, and practices. A preferred time-frame of scrubbing the various fittings is between 15 and 30 seconds. A preferred sterile pad may be a sterile alcohol pad or other sterilizing device.

It will be understood that the removal of the plurality of port caps 250 from any one of the ports (10, 20, 30, 40) or external devices (210, 220, 230, 240) may be performed in any order but there may be a preferred order required for various procedures. For instance in some cases, it may be that an operator is not allowed to remove the port cap 250 located on the discharge port 40 until after all of the other port caps 250 have been removed and the external devices (210, 220, 230) have been installed and the medicine is ready to be injected to the patient. It will also be understood that in some instances, the plurality of port caps 250 will be preinstalled from the manufacturer onto the catheter manifold, but in some cases, the external devices (210, 220, 230, 240) may come preinstalled onto the fittings of the catheter manifold device 200.

It will further be understood that various steps described herein may be taken by various parties. For instance in some cases, the manufacturer may install the port caps 250, and a hospital or pharmacy may remove some of the port caps 250 and install the external devices (210, 220, 230), and finally a nurse or the patient will remove the port cap 250 on the discharge port 40 and install the catheter port 240.

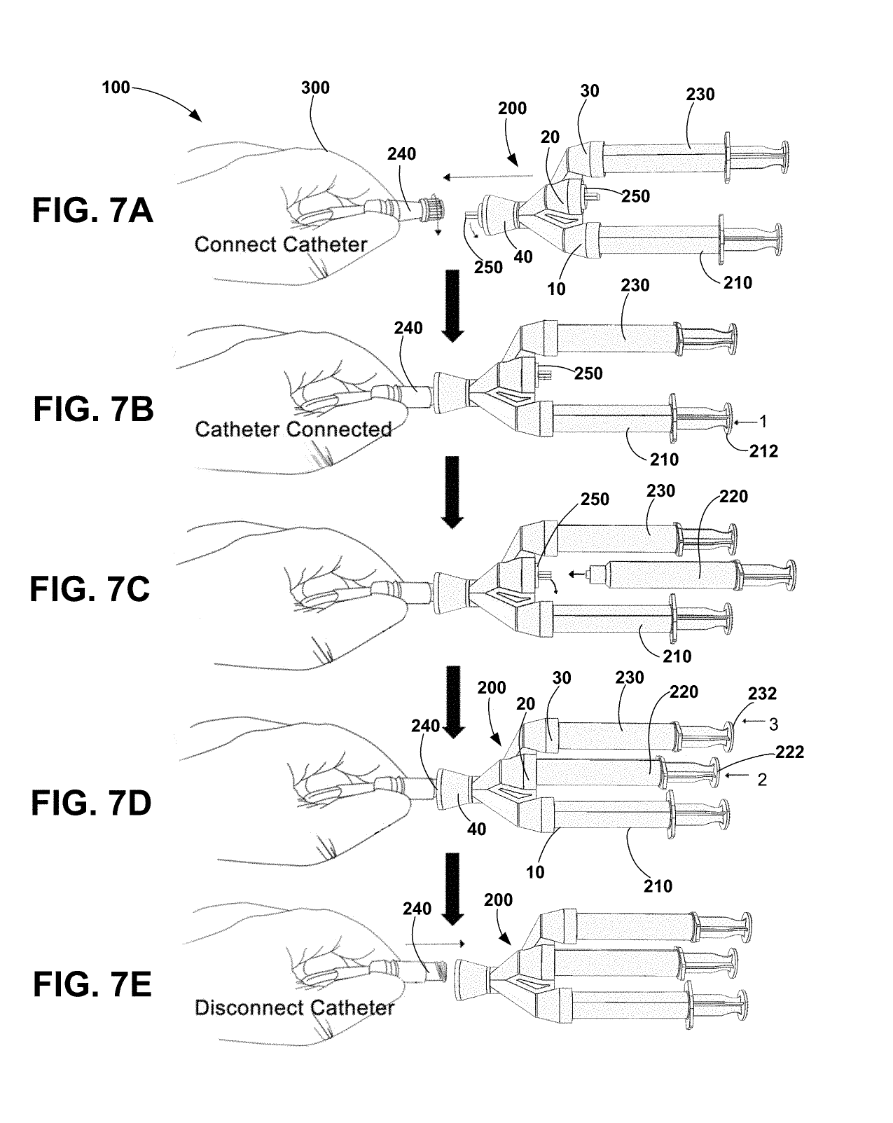

Turning to FIGS. 7A-7E, depicted therein is an alternative exemplary method of using the catheter manifold system 100 which has the wash device 210 and the flush device 230 preinstalled, which may be done by a pharmacy, doctor, hospital, or manufacturer, preferably in a sterile environment.

In FIG. 7A, the operator removes the port cap 250 from the discharge port 40 and removes any protective cap located on the catheter port 240. The operator then connects the catheter port to the discharge port 40 of the catheter manifold device 200 as depicted in FIG. 7B. The operator then administers the first fluid using the wash device 210 connected to the wash port 10. The operator administers the first fluid by pushing the mechanical plunger 212 of the wash device 210, which pushes the first fluid through the wash inlet opening 15 of the wash port 10 and then into the wash channel 14. The first fluid then travels downstream through the wash funnel 17 and through the wash outlet opening 19, into the discharge channel 46, and then through the discharge opening 45, and into the catheter port 240.

Turning to FIG. 7C, the operator 300 removes the port caps 250 from the delivery port 20 and removes any protective cap located on the fittings of the delivery device 220. The operator then connects the delivery device 220 to the delivery port 20.

Next, as depicted in FIGS. 7D and 7E, the operator administers the second fluid and the third fluid similar to that described in the first embodiment in FIGS. 6D and 6E and then disconnects the catheter port.

It will be understood that although the methods described in FIGS. 6A-6E and 7A-7E include various steps taken by an operator, including removal of caps or installation of the external devices. The system could include variations or different ordering of these steps. For instance, the catheter manifold device 200 may come preinstalled either with the plurality of port caps 250, or come preinstalled from a pharmacy, hospital, manufacturer or the like, with the wash device 210, the delivery device 220, and/or the flush device 230. Further, the catheter manifold system 200 could come with a catheter port 240 preinstalled so that all that is required is attaching the catheter line to a patient and administration of the saline and medicine to the patient without the need to scrub or wash any ports.

Depicted in FIGS. 8-9, is an alternative embodiment of a catheter manifold device 500. The catheter manifold device 500 is configured in a “y,” or staggered configuration in contrast to the more linear configuration of the catheter manifold device 200, but has similar structures and operates in a similar manner to that of the catheter manifold device 200. The catheter manifold device 500 includes a flush port 510, a delivery port 520, a wash port 530, and a discharge port 540.

If the specification or claims refer to “an additional” element, that does not preclude there being more than one of the additional element.

It is to be understood that where the claims or specification refer to “a” or “an” element, such reference is not to be construed that there is only one of that element.

It is to be understood that where the specification states that a component, feature, structure, or characteristic “may”, “might”, “can” or “could” be included, that particular component, feature, structure, or characteristic is not required to be included.

It is to be understood that were the specification or claims refer to relative terms, such as “front,” “rear,” “lower,” “upper,” “horizontal,” “vertical,” “above,” “below,” “up,” “down,” “top,” “bottom,” “left,” and “right” as well as derivatives thereof (e.g., “horizontally,” “downwardly,” “upwardly” etc.), such reference is used for the sake of clarity and not as terms of limitation, and should be construed to refer to the orientation as then described or as shown in the drawings under discussion. These relative terms are for convenience of description and do not require that the apparatus be constructed or the method to be operated in a particular orientation. Terms, such as “connected,” “connecting,” “attached,” “attaching,” “join” and “joining” are used interchangeably and refer to one structure or surface being secured to another structure or surface or integrally fabricated in one piece.

The invention presented here reduces contamination risks by reducing the number of scrubbing and action steps required by the SAS method and reducing the risks associated with incidental contact with the infusion system terminations (ports) and catheter port. The invention introduces a manifold-style adapter installed between the port on the catheter and the infusion devices delivering various therapeutic solutions.

Still further, additional aspects of the invention may be found in one or more appendices attached hereto and/or filed herewith, the disclosures of which are incorporated herein by reference as if fully set out at this point.

Methods of the instant disclosure may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Thus, the invention is well adapted to carry out the objects and attain the ends and advantages mentioned above as well as those inherent therein. While the inventive concept has been described and illustrated herein by reference to certain illustrative embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A catheter manifold device comprising:
an inlet port comprising:
an inlet housing having an inlet housing height;
an inlet fitting having an inlet fitting height that is less than the inlet housing height, wherein the inlet fitting is concealed from contamination within the inlet housing;
a discharge port; and
a port channel extending from the inlet fitting to the discharge port.

2. The catheter manifold device of claim 1, further comprising a second inlet port comprising:
a second inlet housing having a second inlet housing height;
a second inlet fitting having a second inlet fitting height that is less than the second inlet housing height, wherein the second inlet fitting is concealed from contamination within the second inlet housing; and
a second port channel extending from the second inlet fitting to the port channel.

3. The catheter manifold device of claim 2, wherein the discharge port comprises:
a discharge housing having a discharge housing height;
a discharge fitting having a discharge fitting height that is less than the discharge housing height, wherein the discharge fitting is concealed from contamination within the discharge housing.

4. The catheter manifold device of claim 3, further comprising a third inlet port comprising:
a third inlet housing having a third inlet housing height;
a third inlet fitting having a third inlet fitting height that is less than the third inlet housing height, wherein the third inlet fitting is concealed from contamination within the third inlet housing; and
a third port channel extending from the third inlet fitting to the discharge port.

5. The catheter manifold device of claim 4, further comprising a plurality of caps, wherein each cap of the plurality of caps comprises:
a cap fitting, wherein the cap fitting is configured to connect to a corresponding one of the inlet, second inlet, discharge, or third inlet fittings;
a cap knob, wherein the cap knob extends above the height of a corresponding one of the inlet, second inlet, discharge, or third inlet housings to allow the cap to be removed without contact to one of the inlet, second inlet, discharge, or third inlet fittings.

6. The catheter manifold device of claim 4, wherein each of the corresponding one of the inlet, second inlet, discharge, or third inlet fittings are capable of being connected to syringe.

6. The catheter manifold device of claim 4, wherein each of the corresponding one of the inlet, second inlet, discharge, or third inlet fittings are capable of being connected to a catheter.

7. A method of administering fluid to a patient, associated with a catheter manifold device, the method comprising the steps of:
connecting a catheter to a discharge port of the catheter manifold device;
administering a first fluid from a first fluid delivery device, wherein the first fluid flows through a first port channel extending from a first inlet port fitting on a first port of the catheter manifold device into the discharge port, and into the catheter; and
administering a second fluid from a second fluid delivery device, wherein the second fluid flows through a second port channel extending from a second inlet port fitting on a second port of the catheter manifold device, into the first port channel, into the discharge port, and into the catheter.

8. The method of claim 7 further comprising the steps of:
removing the first fluid delivery device from the first inlet port fitting;
connecting a third fluid delivery device to the first inlet port fitting;
administering a third fluid from the third fluid delivery device wherein the third fluid flows through the first port channel, into the discharge port, and into the catheter.

9. The method of claim 8, wherein the first fluid is a saline.

10. The method of claim 9, wherein the second fluid is a medicine.

11. The method of claim 10, wherein the third fluid is a saline.

12. The method of claim 11, wherein the first, second, and third fluid delivery devices are syringes.

13. A method of administering fluid to a patient, associated with a catheter manifold device, the method comprising the steps of:
connecting a catheter to a discharge port of the catheter manifold device;
administering a first fluid from a first fluid delivery device, wherein the first fluid flows through a first port channel extending from a first inlet port fitting on a first port of the catheter manifold device, into the discharge port, and into the catheter;
administering a second fluid from a second fluid delivery device, wherein the second fluid flows through a second port channel extending from a second inlet port fitting on a second port of the catheter manifold device, into a second port channel, into the discharge port, and into the catheter;
administering a third fluid from a third fluid delivery device wherein the third fluid flows through a third port channel, into the second port channel, into the discharge port, and into the catheter.

14. The method of claim 13 further comprising the steps of:
removing a discharge port cap from the discharge port fitting prior to connecting the catheter;
removing a first inlet port cap from the first inlet port fitting;
connecting the first fluid delivery device to the first inlet port fitting prior to administering the first fluid;
removing a second inlet port cap from the second inlet port fitting;
connecting the second fluid delivery device to the second inlet port fitting prior to administering the second fluid;
removing a third inlet port cap from the third inlet port fitting; and
connecting the third fluid delivery device to the third inlet port fitting prior to administering the third fluid.

15. The method of claim 13, wherein the first fluid is a saline.

16. The method of claim 15, wherein the second fluid is a medicine.

17. The method of claim 16, wherein the third fluid is a saline.

18. The method of claim 17, wherein the first, second, and third fluid delivery devices are syringes.
